(19) United States
(12) Patent Application Publication
(10) Pub. No.: US 2009/0076625 A1
(43) Pub. Date: Mar. 19, 2009

(54) REINFORCED PROSTHETIC SUSPENSION SLEEVE

(75) Inventors: Mark W. Groves, Columbus, OH (US); Christopher T. Kelley, Grandview Heights, OH (US)

Correspondence Address: STANDLEY LAW GROUP LLP, 6300 Riverside Drive, Dublin, OH, 43017, US

(73) Assignee: The Ohio Willow Wood Company, Mt. Sterling, OH (US)

(21) Appl. No.: 11/855,866

(22) Filed: Sep. 14, 2007

(51) Int. Cl. A61F 2/80

(52) U.S. Cl. 623/34

(57) ABSTRACT

A prosthetic suspension sleeve having reinforcement that prevents or minimizes damage to the suspension sleeve, particularly as a result of abrasion, puncture, or an impact with another object during use. The reinforcement may be selectively located along one or more interior, exterior, or interior and exterior areas of a suspension sleeve. The reinforcement material may be comprised of one or more materials that preferably exhibit resistance to cuts, tears, punctures and/or other damage. In an alternative embodiment, reinforcement may be provided by mixing one or more reinforcing materials into the polymeric material of the suspension sleeve. Combinations of different reinforcement types may be employed.

### BACKGROUND OF THE INVENTIVE FIELD

The present invention is directed to a prosthetic suspension sleeve and, more particularly, to a reinforced prosthetic suspension sleeve having at least one or more areas thereof that are resistant to damage.

Suspension sleeves such as those of interest herein would be well known to one skilled in the art of prosthetics. Such sleeves may also be found in the field of orthotics, where they may be used as or in conjunction with a brace.

Suspension sleeves may be of various design, size and shape. Typically, such sleeves are manufactured from a polymeric material that is substantially air impermeable and is also substantially elastic in nature. Some suspension sleeves, such as those manufactured by The Ohio Willow Wood Company in Mt. Sterling, Ohio, utilize a polymeric material having at least a portion or the entirety of an exterior surface thereof covered with fabric. With respect to these particular suspension sleeves, one or more portions of the interior (limb contacting) surface of the polymeric material may also be covered with fabric.

In the prosthetics field, suspension sleeves are typically worn in conjunction with a prosthetic socket. That is, once an amputee has inserted his/her residual limb into the socket of a prosthesis, a suspension sleeve may be donned to seal the open (proximal) end of the socket. When used in this manner, one end of the suspension sleeve is located to overlie the proximal end of the socket while the other end of the suspension sleeve is located to overlie a portion of the amputee's residual limb (which may be covered by a prosthetic liner). In this manner, air may be prevented from entering or exiting the socket from the proximal end thereof, thereby facilitating creation and maintenance of a vacuum within the socket. The ability to create and maintain vacuum within a socket is important, if not critical, when the associated prosthesis is retained on the residual limb by means of suction suspension.

It can be understood from the foregoing remarks that any holes, cuts, tears or other similar damage to a suspension sleeve may hinder or destroy the ability of the suspension sleeve to seal a socket and ensure vacuum maintenance. Even very small holes can result in loss of vacuum (suction) suspension. It is desirable, therefore, that any such damage be avoided.

Unfortunately, it has been discovered that the design of a typical prosthetic socket has a tendency to cause damage to known suspension sleeves during the use thereof. This problem can occur with any type of prosthesis, including below knee (BK), above knee (AK), knee disarticulation, and upper extremity prostheses.

The proximal end of a prosthetic socket includes an edge or brim that defines the residual limb receiving opening therein. This brim may have varying degrees of sharpness, depending on factors such as the design of the socket, the materials used to make the socket, and the skill of the prosthetist that created the socket. It has been found, however, that even a radiused socket brim can cause damage to an overlying suspension sleeve.

Such damage can result from long-term repeated rubbing of the brim against the sleeve. Another problem, however, is damage caused when a suspension sleeve is impacted with another object while worn by an amputee over his/her prosthetic socket. For example, a BK socket may be impacted with any of various objects during ambulation and other common activities. Such impacts may occur with furniture such as desks or tables during sitting, factory equipment while working, and doors and door frames while entering or exiting a vehicle. Yet another problem is puncturing resulting from contact with a sharp object, such as a nail or the claw's of an animal.

Impact-related damage can often be traced to the pinching of the suspension sleeve between the socket and the impacted object (whether in the brim area, shin area, or otherwise). Because the force of such an impact is often concentrated over a small section of the suspension sleeve (e.g., at the underlying socket brim), damage to the suspension sleeve is not uncommon. In fact, it has been found that such impacts can frequently produce holes, cuts or tears in or through a suspension sleeve. The damage can be significant or almost unnoticeable. However, even a minor hole, cut or tear in a suspension sleeve can prevent or inhibit the generation and/or maintenance of vacuum within an associated prosthetic socket.

As described above, a damaging pinching of a suspension sleeve may occur when the suspension sleeve is trapped between a sharp feature of the prosthetic socket and an impacted object. Conversely, damaging pinching of a suspension sleeve can also occur when the suspension sleeve is trapped between a sharp feature of an impacted object (e.g., the edge of a stair) and a smooth portion of the prosthetic socket (e.g., the shin area). Obviously, such pinching can also occur between two sharp features as well.

Based on the foregoing remarks, it can be understood that there is a need for a prosthetic suspension sleeve that is more resistant to damage, and particularly to abrasion, puncture, and impact damage, than are the suspension sleeves that are currently available. A reinforced prosthetic suspension sleeve of the present invention satisfies this need.

### SUMMARY OF THE GENERAL INVENTIVE CONCEPT

The present invention contemplates reinforced prosthetic suspension sleeves that are better able to resist the damage that may commonly occur during use. Various types of suspension sleeves may be reinforced according to the present invention, such as the fabric-covered suspension sleeves mentioned above. While the present invention is not to be considered limited to the descriptions provided therein, the design and construction of various prosthetic suspension sleeves is described in detail in U.S. Pat. No. 6,406,499, the teachings of which are hereby incorporated by reference herein.

Certain embodiments of a reinforced prosthetic suspension sleeve of the present invention include reinforcing material in one or more areas that are most likely to be subject to damage, such as the damage described above. Particularly, a reinforced prosthetic suspension sleeve of the present invention may include reinforcing material at least in the area thereof that will overly the brim and/or shin area of a prosthetic socket. When reinforcing material is provided in the area of the prosthetic socket brim, the reinforcing material preferably follows, or substantially follows, the path of the socket brim.

Reinforcing material may be provided on a suspension sleeve so as to overlie an entire prosthetic socket brim. Alternatively, reinforcing material may be provided on a suspension sleeve to overlie only that portion of a prosthetic socket brim that is most likely to be impacted with another object (such as along the front and side portions of the socket).

In certain embodiments of the present invention, reinforcing material may be provided as a single unitary section. In alternate embodiments, a plurality of separate reinforcing material sections may be provided. In yet other embodiments of the present invention, a plurality of separate reinforcing material sections may be used in combination with a unitary section of reinforcing material. In any of these embodiments, the reinforcing material(s) may be placed along an interior, exterior, or both an interior and exterior portion of a prosthetic suspension sleeve. A number of different materials may be successfully employed as a reinforcing material.

In other embodiments of a suspension sleeve of the present invention, reinforcement may be accomplished by mixing one or more reinforcing materials into the polymeric material of the suspension sleeve. The reinforcing material(s) remains embedded in the polymeric material upon the curing thereof. The reinforcing material(s) increases the resistance of a suspension sleeve to the type of damage described above, while simultaneously permitting the necessary stretching of the suspension sleeve. Internal reinforcement of the polymeric material may be used alone or in conjunction with the interior and/or exterior reinforcement techniques mentioned above.

A better understanding of the present invention may be gained by reference to the exemplary embodiments described below and shown in the accompanying drawing figures. As would be obvious to one skilled in the art, however, a prosthetic suspension sleeve of the present invention may also occur in a multitude of other embodiments, the exhaustive nature of which cannot be fully described and/or shown in detail herein.

### BRIEF DESCRIPTION OF THE DRAWINGS

In addition to the features mentioned above, other aspects of the present invention will be readily apparent from the following descriptions of the drawings and exemplary embodiments, wherein like reference numerals across the several views refer to identical or equivalent features, and wherein:

FIG. 1 illustrates the basic shape of a common prosthetic socket—in this case, a below knee prosthetic socket;

FIG. 2 depicts, in partial cutaway, an exemplary embodiment of a reinforced prosthetic suspension sleeve of the present invention, with the sleeve turned inside out;

FIG. 3 shows the prosthetic suspension sleeve of FIG. 2 donned over a residual limb and the prosthetic socket of FIG. 1;

FIG. 4 illustrates, in partial cutaway, another exemplary embodiment of a reinforced prosthetic suspension sleeve of the present invention, with the sleeve turned inside out;

FIG. 5 depicts, in partial cutaway, yet another exemplary embodiment of a reinforced prosthetic suspension sleeve of the present invention;

FIG. 6 shows the prosthetic suspension sleeve of FIG. 5 donned over a residual limb and prosthetic socket; and

FIG. 7 illustrates, in partial cutaway, still another exemplary embodiment of a reinforced prosthetic suspension sleeve of the present invention, wherein said reinforcement is accomplished by mixing one or more reinforcing materials into the polymeric material of the suspension sleeve.

### DETAILED DESCRIPTION OF EXEMPLARY EMBODIMENT(S)

As described above, a prosthetic suspension sleeve (“suspension sleeve” or “sleeve”) of the present invention can be used with various types of prostheses—whether upper extremity or lower extremity. However, for purposes of clarity, specific exemplary embodiments of a suspension sleeve of the present invention are described in detail herein only with respect to their use with a below-knee (BK) prosthesis.

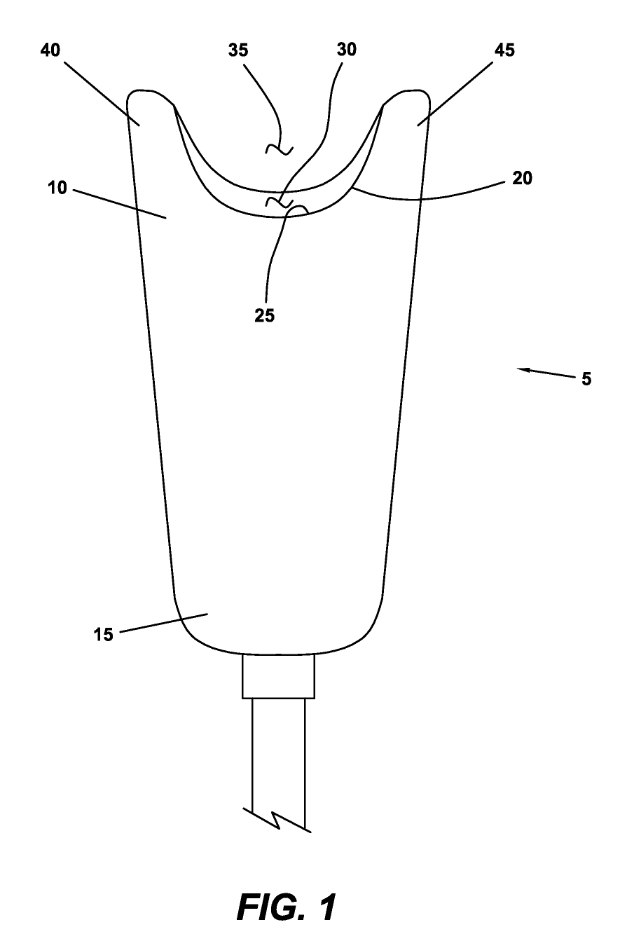

To that end, a typical BK prosthetic socket 5 is illustrated in FIG. 1. As shown, the BK prosthetic socket 5 is formed as a substantially hollow shell having an open (proximal) end 10 into which an amputee's residual limb is inserted, and a closed (distal) end 15 to which other components of the associated prosthesis (typically, a pylon) are generally connected.

The proximal end 10 of the socket 5 terminates at an edge or brim 20 that borders the opening 25 therein. It is this socket brim 20 that has been found particularly capable of causing premature damage to an overlying suspension sleeve upon impact of the prosthetic socket with another object.

In the case of a BK prosthetic socket, it can be seen that the proximal end 10 and, thus, the brim 20, is not of simplistic shape. Rather, this BK prosthetic socket 5, and most other BK prosthetic sockets, have a proximal end that is designed to accommodate movement of the amputee's knee joint. As such, the proximal end 10 of the BK prosthetic socket 5 includes an anterior and posterior recess 30, 35 that permits substantially uninhibited flexion of the knee joint.

While the anterior and posterior recesses 30, 35 are provided to permit proper knee flexure, the lateral portions 40, 45 of the proximal end 10 of the socket 5 can be seen to extend farther upward. These projecting lateral portions 40, 45 are commonly referred to as socket “ears”. These socket ears help to secure the socket 5 to the residual limb and to provide stability during ambulation.

Any portion of the socket brim 20 is theoretically capable of damaging an overlying suspension sleeve upon impact of the prosthetic socket with another object. Thus, it is possible that certain embodiments of a reinforced suspension sleeve of the present invention may be reinforced around the entire circumference thereof. However, since it is more probable that only the anterior and/or lateral portions of the prosthetic socket brim 20 will be subjected to impact incidents, the exemplary embodiments described below are reinforced in only those areas.

Suspension sleeves of the present invention are substantially tubular in nature and have two open ends. As used herein, the term “tubular” is intended to denote only that a sleeve is a continuous hollow structure of some length. As would be understood by one skilled in the art, a suspension sleeve of the present invention may have a generally circular cross section when in use, although the flexible nature thereof also permits a sleeve to conform to other cross-sectional shapes. Suspension sleeves of the present invention may have a taper. When present, the degree of taper may vary. Other suspension sleeve embodiments may be substantially cylindrical (i.e., may have a substantially uniform cross-sectional diameter along the entire length). Yet other embodiments may have a larger cross-sectional diameter at or near a mid-point than at each end. These designs and others would be well known to one of skill in the art, and all are considered to be “tubular,” as well as falling within the scope of the present invention.

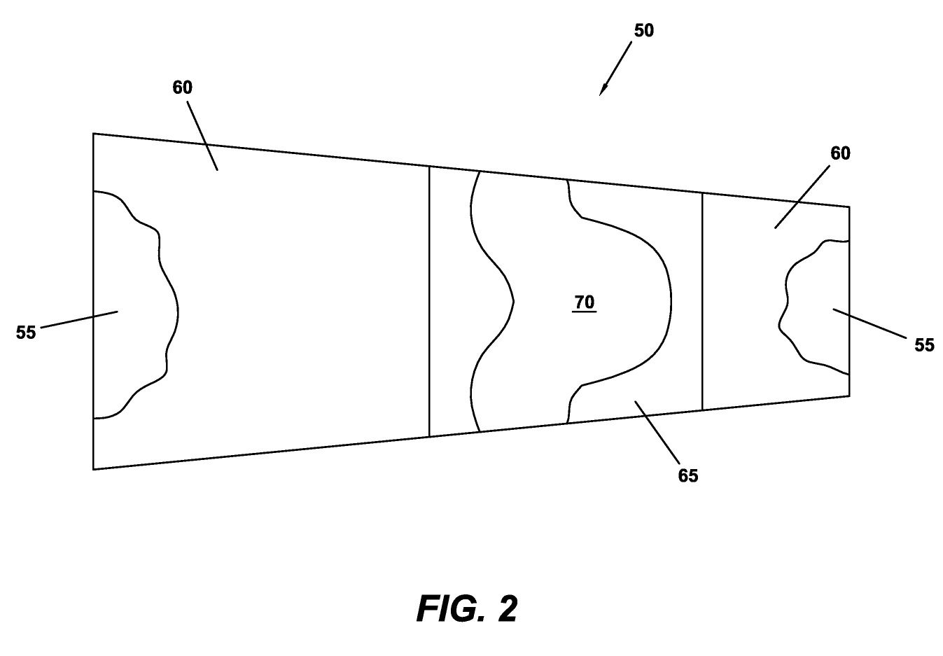

One embodiment of a suspension sleeve 50 of the present invention is shown in FIG. 2. This particular suspension sleeve 50 is shown to have an optional taper.

The suspension sleeve 50 is shown in an inside out condition so as to better illustrate the design and construction thereof. As shown, the suspension sleeve 50 includes a fabric material 55 that covers the exterior of the suspension sleeve when the suspension sleeve is in a normal (i.e., right side out) orientation. A flexible polymeric material 60 resides on an interior surface of the fabric material. A portion of the polymeric material 60 will overlap an amputee's residual limb when the suspension sleeve 50 is in use. This portion of the polymeric material may be in contact with the skin of the amputee's residual limb and/or a prosthetic liner covering the residual limb. Another portion of the polymeric material 60 will overlap and be in contact with the exterior of a prosthetic socket when the suspension sleeve 50 is in use.

This particular suspension sleeve 50 also includes an optional interior band (cuff) 65. The interior cuff 65 may be formed of a fabric material, which may be the same as or dissimilar to the fabric material 55 that covers the exterior of the suspension sleeve 50. The cuff 65 can allow for easier manipulation of the suspension sleeve over the residual limb and prosthetic socket, and may also produce an area of reinforcement. The cuff 65 may be located at various points along the length of the suspension sleeve 50, but is preferably located at or near the point where the suspension sleeve will overlap the brim of the prosthetic socket when in use. When present on a sleeve of the present invention, a cuff may fully circumvolve the interior of the sleeve, or may cover only a section of the sleeve interior.

In this particular embodiment, a section of reinforcing material 70 is affixed to the exposed surface of the cuff 65. In other similar embodiments, a section of reinforcing material 70 may reside between the interior surface of the polymeric material 60 and the mating surface of the overlying cuff 65. In embodiments without an interior cuff, the section of reinforcing material 70 may be affixed directly or indirectly to the exposed interior surface of the polymeric material 60.

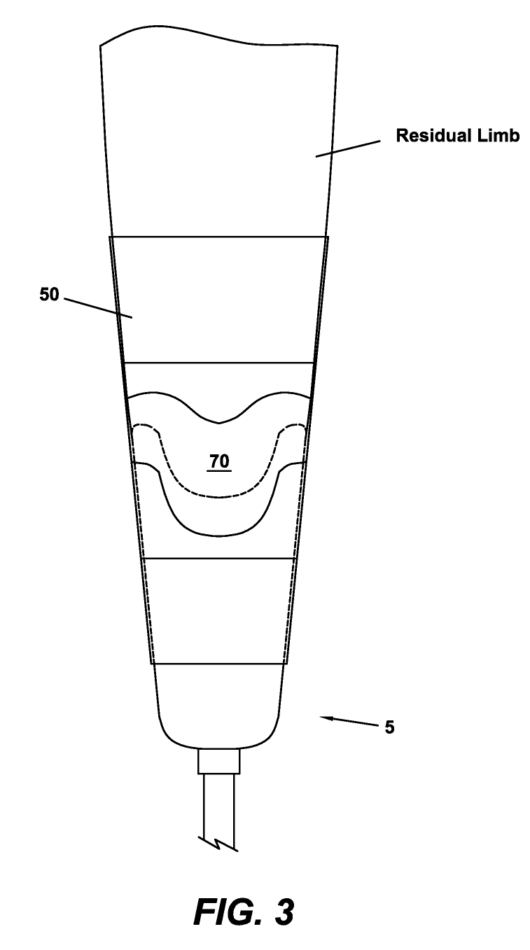

This section of reinforcing material 70 is of a shape that substantially corresponds to the path defined by the brim 20 of the prosthetic socket 5 depicted in FIG. 1. More specifically, when the suspension sleeve 50 is properly donned over the prosthetic socket 5 and amputee's residual limb, as shown in FIG. 3, the section of reinforcing material 70 will substantially trace and overlie at least the anterior portion of the brim 20 of the prosthetic socket. As shown, this section of reinforcing material 70 will also cover at least a portion of the brim 20 present on each socket ear. In other embodiments, the section of reinforcing material could cover a lesser or greater portion of the socket brim. For example, a section of reinforcing material could be provided to overlie the entirety of a prosthetic socket brim.

The section of reinforcing material 70 is preferably of sufficient size to ensure that the associated portion of the socket brim 20 will be covered when the suspension sleeve is properly, or substantially, properly positioned over the prosthetic socket and residual limb. Preferably, the section of reinforcing material is sufficiently large so as not to require perfect positioning, but not so large as to inhibit the sealing ability or flexibility of the suspension sleeve.

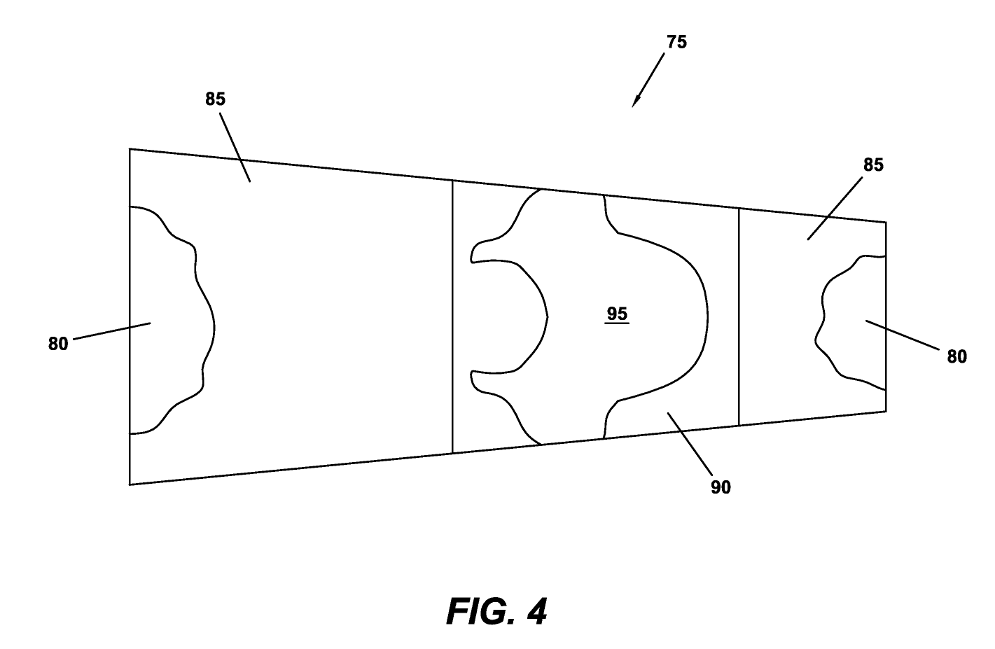

An alternate embodiment of a prosthetic suspension sleeve 75 of the present invention can be observed in FIG. 4. As with the suspension sleeve 50 of FIG. 2, this suspension sleeve 75 also has an optional taper and is shown in an inside out condition so as to better illustrate the design and construction thereof.

The suspension sleeve 75 again includes a fabric material 80 that covers its exterior when the suspension sleeve is in a normal (i.e., right side out) orientation. A flexible polymeric material 85 resides on an interior surface of the fabric material. A portion of the polymeric material 85 will overlap an amputee's residual limb when the suspension sleeve 75 is in use. This portion of the polymeric material may be in contact with the skin of the amputee's residual limb and/or a prosthetic liner covering the residual limb. Another portion of the polymeric material 85 will overlap and be in contact with the exterior of a prosthetic socket when the suspension sleeve 75 is in use.

The suspension sleeve 75 also includes an optional interior cuff 90, which may be similar in size and construction to the cuff 65 of the suspension sleeve 50 shown in FIG. 2. The cuff 90 may again be located at various points along the length of the suspension sleeve 75, but is preferably located at or near the point where the suspension sleeve will overlap the brim of the prosthetic socket when in use.

A section of reinforcing material 95 is affixed to the exposed inner surface of the cuff 90. In other similar embodiments, a section of reinforcing material 95 may reside between the interior surface of the polymeric material 85 and the mating surface of the overlying cuff 90. In embodiments without an interior cuff, the section of reinforcing material 95 may be affixed directly or indirectly to the exposed interior surface of the polymeric material 85. As can be observed by a comparison of FIG. 2 and FIG. 4, this section of reinforcing material 95 is of a shape that substantially corresponds to the path defined by a prosthetic socket brim that is somewhat dissimilar in shape to the brim 20 of the prosthetic socket 5 depicted in FIG. 1.

As with the suspension sleeve 50 of FIG. 2, proper donning of this suspension sleeve 75 will result in the section of reinforcing material 95 substantially tracing and overlying the anterior portion of an underlying socket brim as well as at least a portion of the brim present on each socket ear of a prosthetic socket. Of course, in other embodiments, the section of reinforcing material could cover a lesser or greater portion of the socket brim. For example, a suspension sleeve could be provided with a section of reinforcing material that will overlie the entirety of a prosthetic socket brim when in use.

In addition to the aforementioned benefits, the “manta ray” shape of the section of reinforcing material 95 shown in FIG. 4 aids in proper placement over the patella of the residual limb and reduces pressure on the amputee's knee when the residual limb is in flexion. The protuberances at the top of the reinforcing material section 95 also help to minimize bunching of the fabric material behind the knee (by pulling the fabric material upward) and assist with retention of the reinforcing material section in the preferred location.

As described with respect to the suspension sleeve 50 of FIG. 2, the section of reinforcing material 95 present on this embodiment of the suspension sleeve 75 is preferably of sufficient size to ensure that the designated portion of an associated socket brim will be covered when the suspension sleeve is properly, or substantially, properly positioned over the prosthetic socket and residual limb. Preferably, the section of reinforcing material is sufficiently large so as not to require perfect positioning, but not so large as to inhibit the sealing ability or flexibility of the suspension sleeve.

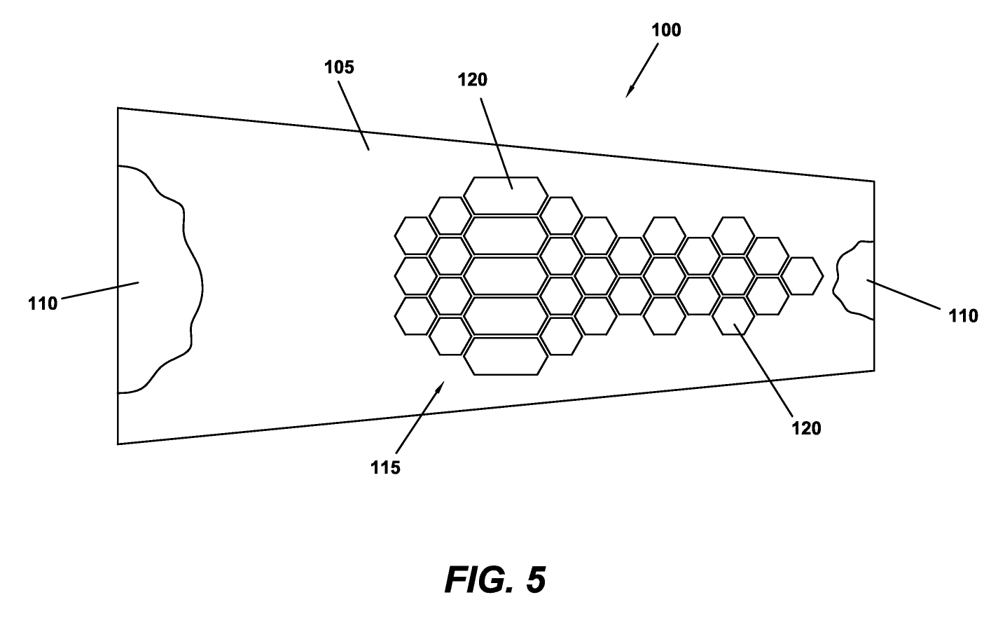

Yet another embodiment of a suspension sleeve 100 of the present invention is illustrated in FIG. 5. As with the suspension sleeves 50, 75 of FIG. 2 and FIG. 4, this suspension sleeve 100 also has an optional taper. Unlike the suspension sleeves 50, 75 of FIG. 2 and FIG. 4, however, this suspension sleeve 100 is shown in a normal (i.e., right side out) orientation.

The suspension sleeve 100 again includes a fabric material 105 that covers its exterior. A flexible polymeric material 110 resides along an interior surface of the fabric material 105. A portion of the polymeric material 110 will overlap and be in contact with the skin of an amputee's residual limb and/or a prosthetic liner covering the amputee's residual limb when the suspension sleeve 100 is in use. Another portion of the polymeric material 110 will be in contact with the exterior of a prosthetic socket when the suspension sleeve 100 is in use.

In this particular embodiment, a reinforced area 115 of the suspension sleeve 100 is produced by affixing a number of individual pieces of reinforcing material 120 to the exterior surface of the fabric material 105. In other embodiments, individual pieces of reinforcing material 120 may be located between the exterior surface of the polymeric material 110 and the mating surface of the fabric material 105. A combination of these two reinforcing techniques may also be employed.

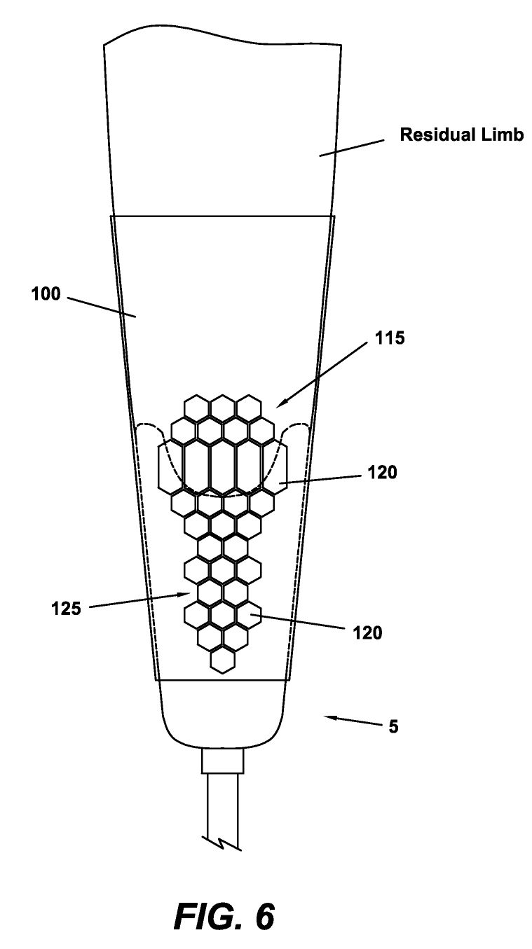

As can be observed in FIG. 6, the overall area of the suspension sleeve covered by the individual pieces of reinforcing material 120 substantially corresponds to an area thereof that at least overlies the brim of a prosthetic socket when the suspension sleeve is worn by an amputee. Thus, as with the suspension sleeves 50, 75 of FIG. 2 and FIG. 4, proper donning of this suspension sleeve 100 will result in the section of reinforcing material 120 substantially overlying at least the anterior portion of the brim present on a prosthetic socket. Of course, in other embodiments, the section of reinforcing material could cover a lesser or greater portion of the socket brim. For example, a section of reinforcing material could be provided to overlie the entirety of a prosthetic socket brim.

It is contemplated that other areas of the suspension sleeve may also be protected. For example, as shown in FIG. 6, the section of reinforcing material 115 may extend along what will be a shin area 125 of the suspension sleeve 100 when the suspension sleeve is donned over a prosthetic socket.

The reinforced area 115 of the suspension sleeve 100 is preferably of sufficient size to ensure that the designated portion of an associated socket brim (or other socket area) will be covered when the suspension sleeve is properly, or substantially properly, positioned over the prosthetic socket and residual limb. Preferably, the reinforced area is sufficiently large so as not to require perfect positioning of the suspension sleeve, but not so large as to inhibit the sealing ability or flexibility of the suspension sleeve.

Although individual pieces of reinforcing material 120 are used in this embodiment of the suspension sleeve 100, it should be realized that a reinforced area on the exterior of a suspension sleeve may also be created by affixing a unitary section of reinforcing material thereto. For example, unitary sections 70, 90 of reinforcing material such as those shown in FIGS. 2 and 4 may be used. However, the exterior surface of a suspension sleeve is generally subjected to a greater degree of stretch than is the interior surface. Consequently, it has been determined that affixing a number of individual pieces of reinforcing material 120 to the exterior surface of a suspension sleeve allows for more uninhibited stretching of the exterior surface thereof, while still providing for adequate reinforcement.

The individual pieces of reinforcing material 120 shown in FIG. 5 are of two related but dissimilar shapes. Obviously, a virtually unlimited variety of shapes may be used, and a multitude of different shape combinations may be employed. Preferably but not essentially, however, the shape(s) of the individual pieces of reinforcing material are selected so as to minimize the amount of exposed sleeve material residing therebetween. In this manner, maximum protection is afforded by the reinforcing material while permitting the necessary stretching of the suspension sleeve.

The sections/pieces of reinforcing material employed in interior and/or exterior reinforced embodiments of the present invention may be affixed to the suspension sleeve in a number of ways. As may be described in more detail below, reinforcing material(s) may generally be affixed to a suspension sleeve of the present invention by: curing or molding in place; adhesive bonding; radio frequency (RF) welding; heat staking; stitching; insertion into a pocket formed in/on the sleeve; and/or by any combination thereof. Reinforcing materials could also be affixed in a temporary manner, such as with hook-and-loop material. Obviously, the particular affixation technique(s) employed may depend largely on the composition of the reinforcing material used, and the above list of affixing techniques is not to be considered as in any way limiting the affixing techniques that may be used in conjunction with the present invention.

The reinforcing materials employed with the interior and exterior reinforcement techniques discussed above may be of various composition. For example, it has been found that polyurethane rubber, such as milled polyurethane rubber provides for good results, when used as an interior and/or exterior surface reinforcing material. Interior and exterior surface reinforcing materials of the present invention may also be of other compositions and may be affixed/applied by a number of different techniques, such as those previously described. For example, reinforcing materials of the present invention may also be specifically constructed from/affixed by: silicone rubber, such as milled silicone rubber; two part liquid silicone rubber; two part polyurethane rubber; polyurethane sheet; silicone sheet; one part moisture cured silicone; shear thickening material encapsulated in small compartments; shear thickening molded polymer; sheet materials of various thicknesses retained in a sleeve pocket(s), such as—various types of ethylene vinyl acetate (EVA),urethane and other types of foam pads, flexible urethane and other types of flexible polymer films, rigid polycarbonate and other types of rigid films, thermoformed polycarbonate sheet and other types of thermoformed sheet, thermoformed flexible urethane sheet with a foam backer, thermoformed O&P splint materials with or without a foam backer, injection molded EVA foam and other types of injection molded foams, neoprene pads, neoprene foam pads, other types of rubber and foam rubber pads, a stretch fabric backed array of (e.g., hex shaped) foam panels, viseoelastic foam, viseoelastic foam encapsulated in a dielectrically sealed thermoformed urethane film and fabric pillow, other types of foam encapsulated in a dielectrically sealed thermoformed urethane film and fabric pillow, overlaid/overlapping patterns of urethane film panels, overlaid/overlapping patterns of other types of flexible or rigid film panels, Dow active protection system pads, and D₃O sheet and pads and fabric paints; knit, woven or braided fabric (e.g., Kevlar®-based cut resistant materials); impact absorbing polymers (e.g., Sorbothane® or other impact gels); combinations of foam sheets with urethane films in pockets; combinations of milled urethane islands with an underlayment of Kevlar® and/or other cut-resistant fabrics cured to a fabric sleeve; combinations of milled urethane islands with an underlayment of Kevlar® and/or other cut-resistant fabrics and a thin layer of foam cured to a fabric sleeve; heat-activated adhesive barrier fabric (e.g., wet suit repair fabric) with cast urethane(s) islands bonded to fabric; heat-activated adhesive barrier fabric with self-skinning urethane foam(s) islands bonded to fabric; heat-activated adhesive barrier fabric with cast silicone islands bonded to fabric; urethane resin(s) islands directly cast to fabric; self-skinning urethane foam(s) islands directly cast to fabric; silicone resin(s) islands directly cast to fabric; cast urethane resin islands attached via liquid, solvent-activated, pressure sensitive, moisture and/or heat-activated film adhesive; flexible and rigid urethane films; thermoformed rigid plastic sheet material; other puncture resistant fabrics; EVA foams and resin-transferred self-skinning foam urethanes, urethane and silicones; poured urethanes and silicone islands applied by selective masking with tapes, waxes, stencils, or by use of liquid resists and/or cured milled urethane as resin dams; and cast urethane islands and self-skinning foam urethane islands flash laminated to fabric. Any number of combinations of these and other suitable materials and/or affixation techniques may be employed.

While a variety of reinforcing material composition(s) may obviously be used, it is preferred, but not essential, that the selected reinforcing material(s) have a greater resistance to cutting, tearing, puncturing and other similar damage than does the basic material(s) of the suspension sleeve. It is also possible that one or more additional layers of one or more suspension sleeve materials might function acceptably when used as a reinforcing material.

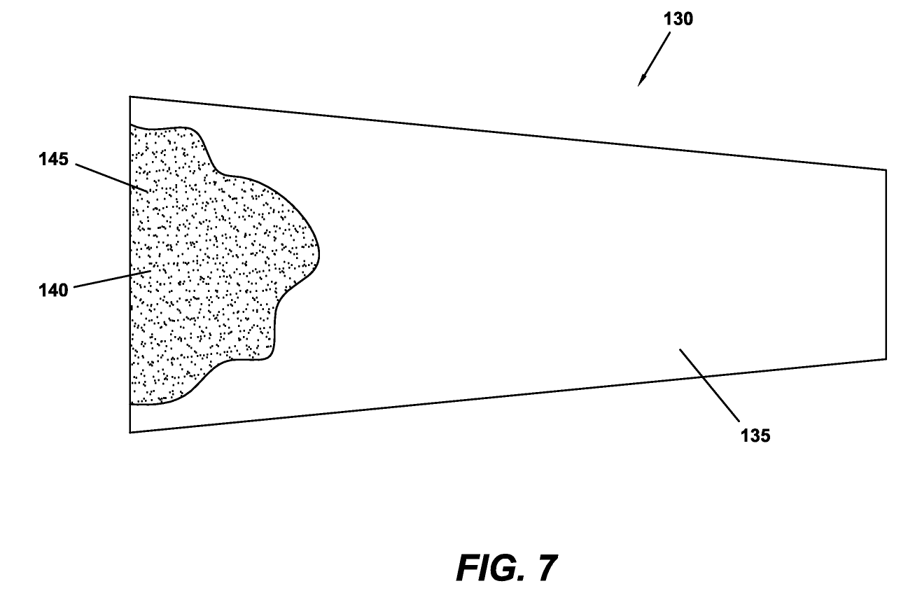

Still another embodiment of a suspension sleeve 130 of the present invention can be observed by reference to FIG. 7. As with the suspension sleeves 50, 75, 100 of FIGS. 2, 4 and 5, this suspension sleeve 130 also has an optional taper. Unlike the suspension sleeves 50, 75, 100 of FIGS. 2, 4 and 5, however, this suspension sleeve 130 does not employ selective surface reinforcement. Rather, this embodiment of the suspension sleeve 125 is reinforced by mixing one or more reinforcing materials into the polymeric material that forms at least a portion of the suspension sleeve.

As can be seen in FIG. 7, the suspension sleeve 130 again includes a fabric material 135 that covers its exterior. A flexible polymeric material 140 resides on an interior surface of the fabric material 135. A portion of the polymeric material 140 will be in contact with the skin of an amputee's residual limb and/or a prosthetic liner covering the amputee's residual limb when the suspension sleeve 130 is in use. Another portion of the polymeric material 140 will be in contact with the exterior of a prosthetic socket when the suspension sleeve 130 is in use.

In this embodiment, the polymeric material 140 itself contains one or more reinforcing materials 145. More specifically, one or more reinforcing materials are mixed into the polymeric material while the polymeric material is in a liquid or semi-liquid state. Consequently, the reinforcing material is subsequently trapped in the cured polymeric material portion of the suspension sleeve. In certain embodiments of the present invention, only desired sections of the polymeric material may be reinforced in this manner. In other embodiments, the entirety of the polymeric material may be reinforced in this manner.

The reinforcing materials used in such an embodiment are preferably selected to resist abrasion, cutting, tearing, puncturing and other such damage as described above, without unduly limiting the elastic nature of the polymeric material. Various reinforcing materials may be used for this purpose. For example, and without limitation, acceptable reinforcement of the polymeric material of such an embodiment may be accomplished by adding cotton flock, pulped natural or synthetic materials having short and/or long fibers, and/or powders comprising talc, fumed silica (e.g., Cabosil), clay, glass, and plastic microspheres. Kevlar® pulp has been found to work particularly well when used for this purpose. Additionally, a stretch mesh material may be optionally embedded in the polymeric material to help compartmentalize punctures.

In any embodiment of a suspension sleeve of the present invention that makes use of selective reinforcement (e.g., see FIGS. 2, 4 and 5), it is possible that more than one reinforcing materials may be employed and/or more than one layer of the same reinforcing material may be used. For example, sections or pieces of similar or dissimilar reinforcing materials may be affixed to a suspension sleeve in an overlaid fashion. The sections or pieces of reinforcing material may be of the same size and shape, or of different size and/or shape. Various combinations of reinforcing materials may be used, and different reinforcing materials may be affixed to a suspension sleeve in varying order.

It is to be understood that a suspension sleeve of the present invention can also employ combined reinforcement techniques. For example, both selective interior and exterior reinforcement may be provided (e.g., a combination of the embodiments shown in FIGS. 2 and 5). When combined, selective interior and exterior reinforcement may be used to protect the same or different portions of a suspension sleeve. For example, interior and exterior areas of reinforcement may both protect a portion of a suspension sleeve that will overlie an anterior section of a prosthetic brim. Alternatively, one area of reinforcement may protect an anterior portion while the other area(s) protects a posterior or lateral portion(s). Selective interior and/or exterior reinforcement may also be used in conjunction with a reinforced polymeric material (e.g., a combination of the embodiments shown in FIGS. 2 and/or 5 with that of FIG. 7).

It should also be understood that selective reinforcement of a suspension sleeve of the present invention is not limited to only the area thereof that will overlie a prosthetic socket brim while in use. Rather, selective reinforcement can be located at virtually any area of a suspension sleeve that may be subject to damage during use. Thus, certain embodiments of a suspension sleeve of the present invention may have an area(s) of reinforcement material in addition to, or in lieu of, the area that will overlie a prosthetic socket brim (such as in an area that will overlie the shin area of a prosthetic socket when in use).

While only fabric-covered suspension sleeves have been specifically disclosed in the provided exemplary embodiments, it is to be further understood that the present invention also applies to suspension sleeves without a fabric covering. That is, reinforcement material(s) could also be applied according to the present invention directly to suspension sleeves formed entirely of polymeric material. Similarly, reinforcing materials could be mixed into the polymeric materials used to form non-fabric covered suspension sleeves. The same would also obviously apply to suspension sleeves that are only partially fabric-covered, that are fabric-covered on both their exterior and interior surfaces, or that have only one or more interior fabric sections or fabric cuffs and no exterior fabric.

As would be evident from a reading of the foregoing description, and as would be obvious to one of skill in the art, selectively reinforced suspension sleeve embodiments of the present invention may have reinforcement areas of widely varying shape and/or size. As previously explained, the shape and/or size of a selective reinforcement area provided to protect a suspension sleeve from damage related to an underlying prosthetic socket brim may vary considerably depending on the shape thereof. That is, the variation in the shape of an interior and/or exterior area of reinforcing material may be directly related to the variation in shape of the brim of the proximal end of the prosthetic socket with which the associated suspension sleeve will be used.

When the brim of a prosthetic socket traces a circuitous path (e.g., like that shown in FIG. 1), the area of reinforcing material on an associated suspension sleeve may have a more complex shape. When the brim of a prosthetic socket traces a simplistic path, the area of reinforcing material on an associated suspension sleeve may have a more basic shape. As such, one skilled in the art should realize that the exemplary reinforcing material sections/pieces/areas described and/or shown herein are presented for purposes of illustration only, and nothing herein is to be interpreted as limiting the present invention to any particular shape or size thereof. Rather, the present invention contemplates that reinforcement sections, pieces, and/or areas of any size and/or shape necessary to provided for adequate reinforcement of a suspension sleeve may be employed. The reinforcement sections, pieces, and/or areas could be of standard shapes and sizes, or could be customized for individual applications.

Therefore, while certain embodiments of the present invention are described in detail above, the scope of the invention is not to be considered limited by such disclosure, and modifications are possible without departing from the spirit of the invention as evidenced by the following claims:

What is claimed is:

1. A reinforced prosthetic suspension device, comprising:
an open-ended sleeve of polymeric material having an interior surface and an exterior surface, one end of said sleeve for overlapping an open end of a prosthetic socket when said sleeve is worn by an amputee, the other end of said sleeve for overlapping a portion of an amputee's residual limb when said sleeve is worn by an amputee;
at least one section of reinforcing material located on said sleeve so as to overlie at least a portion of said prosthetic socket when said sleeve is worn by said amputee;
wherein said at least one section of reinforcing material includes at least one material that exhibits a greater resistance to abrasion, cutting, and puncturing than does said polymeric material; and
wherein said sleeve is capable of forming an air-tight seal over said open end of said prosthetic socket when worn.

2. The reinforced prosthetic suspension device of claim 1, further comprising a fabric covering overlying and affixed to said exterior surface of said polymeric material.

3. The reinforced prosthetic suspension device of claim 2, wherein said reinforcing material is affixed to an exposed surface of said fabric covering.

4. The reinforced prosthetic suspension device of claim 2, wherein said reinforcing material resides between said exterior surface of said polymeric material and a mating surface of said fabric covering.

5. The reinforced prosthetic suspension device of claim 1, further comprising at least one section of fabric material residing on said interior surface of said polymeric material.

6. The reinforced prosthetic suspension device of claim 5, wherein said reinforcing material is affixed to an exposed surface of said fabric material.

7. The reinforced prosthetic suspension device of claim 5, wherein said reinforcing material resides between said interior surface of said polymeric material and a mating surface of said fabric covering.

8. The reinforced prosthetic suspension device of claim 1, wherein said at least one section of reinforcing material is located on said sleeve so as to overlie at least a portion of a brim of said prosthetic socket.

9. The reinforced prosthetic suspension device of claim 8, wherein said at least one section of reinforcing material substantially traces at least a portion of the path defined by said brim of said prosthetic socket.

10. The reinforced prosthetic suspension device of claim 1, wherein said at least one section of reinforcing material is located on said sleeve so as to overlie a section of a shin portion of said prosthetic socket when said sleeve is worn by said amputee.

11. The reinforced prosthetic suspension device of claim 1, wherein said at least one section of reinforcing material comprises a plurality of individual, spaced apart pieces of reinforcing material.

12. The reinforced prosthetic suspension device of claim 1, wherein said at least one section of reinforcing material includes more than one layer of one or more types of reinforcing material.

13. The reinforced prosthetic suspension device of claim 1, wherein said at least one section of reinforcing material is selected from one or more of the group consisting of a polyurethane rubber, a shear thickening material, and a stretch-fabric backed array of foam pads.

14. The reinforced prosthetic suspension device of claim 1, further comprising at least one reinforcing material substantially encapsulated within said polymeric material.

15. A reinforced prosthetic suspension sleeve, comprising:
a tubular, open-ended polymeric material covering having an interior surface and an exterior surface, one end of said covering for overlapping an open end of a prosthetic socket when said sleeve is worn by an amputee, the other end of said covering for overlapping a portion of an amputee's residual limb when said sleeve is worn by an amputee;
a fabric material overlying and affixed to said exterior surface of said polymeric material;
a fabric band affixed to said interior surface of said covering; and
at least one section of reinforcing material associated with said fabric band and located so as to overlie at least a portion of a brim of said prosthetic socket when said sleeve is worn by said amputee;
wherein said at least one section of reinforcing material includes at least one material that exhibits a greater resistance to abrasion, cutting, and puncturing than does said polymeric material; and
wherein said sleeve is capable of forming an air-tight seal over said open end of said prosthetic socket when worn.

16. The reinforced prosthetic suspension sleeve of claim 15, wherein said reinforcing material is affixed to an exposed surface of said fabric band.

17. The reinforced prosthetic suspension sleeve of claim 15, wherein said reinforcing material resides between said interior surface of said polymeric material and a mating surface of said fabric band.

18. The reinforced prosthetic suspension sleeve of claim 15, further comprising at least one section of reinforcing material affixed to an exterior surface of said fabric material, said at least one section of reinforcing material located so as to overlie at least a portion of said brim of said prosthetic socket when said sleeve is worn by said amputee.

19. A reinforced prosthetic suspension sleeve, comprising:
a tubular, open-ended polymeric material covering having an interior surface and an exterior surface, one end of said covering for overlapping an open end of a prosthetic socket when said sleeve is worn by an amputee, the other end of said covering for overlapping a portion of an amputee's residual limb when said sleeve is worn by an amputee;
a fabric material overlying and affixed to said exterior surface of said polymeric material; and
at least one section of reinforcing material associated with said fabric material and located so as to overlie at least a portion of a brim of said prosthetic socket when said sleeve is worn by said amputee;
wherein said at least one section of reinforcing material includes at least one material that exhibits a greater resistance to abrasion, cutting, and puncturing than does said polymeric material; and
wherein said sleeve is capable of forming an air-tight seal over said open end of said prosthetic socket when worn.

20. The reinforced prosthetic suspension sleeve of claim 19, wherein said reinforcing material is affixed to an exposed surface of said fabric material.

21. The reinforced prosthetic suspension sleeve of claim 19, wherein said reinforcing material resides between said exterior surface of said polymeric material and a mating surface of said fabric material.

22. The reinforced prosthetic suspension sleeve of claim 19, further comprising a fabric band residing on said interior surface of said polymeric material, said fabric band having at least one section of reinforcing material affixed to an exposed surface thereof, said at least one section of reinforcing material located so as to overlie at least a portion of said brim of said prosthetic socket when said sleeve is worn by said amputee.

23. A reinforced prosthetic suspension sleeve, comprising:
a tubular, open-ended polymeric material covering having an interior surface and an exterior surface, one end of said covering for overlapping an open end of a prosthetic socket when said sleeve is worn by an amputee, the other end of said covering for overlapping a portion of an amputee's residual limb when said sleeve is worn by an amputee;
a fabric material overlying and affixed to said exterior surface of said polymeric material; and
at least one reinforcing material substantially encapsulated within said polymeric material;
wherein said at least one reinforcing material includes at least one material that exhibits a greater resistance to abrasion, cutting, and puncturing than does said polymeric material; and
wherein said sleeve is capable of forming an air-tight seal over said open end of said prosthetic socket when worn.

24. The reinforced prosthetic suspension device of claim 23, wherein said reinforcing material is selected from the group consisting of cotton flock, pulped natural material, pulped synthetic material, talc powder, fumed silica powder, clay powder, glass powder, and plastic microspheres.

25. The reinforced prosthetic suspension device of claim 23, further comprising a fabric band affixed to said interior surface of said polymeric material, said fabric band having at least one section of reinforcing material associated therewith, said at least one section of reinforcing material located so as to overlie at least a portion of a brim of said prosthetic socket when said sleeve is worn by said amputee.
